(19) United States
(12) Patent Application Publication
(10) Pub. No.: US 2007/0140992 A1
(43) Pub. Date: Jun. 21, 2007

(54) Taste masking of essential oils using a hydrocolloid

(76) Inventors: Lynn Schick, Columbia, NJ (US); Andre Soshinsky, Randolph, NJ (US); Constantine Georgiades, East Brunswick, NJ (US)

Correspondence Address: Johnson & Johnson, 201 TABOR ROAD, MORRIS PLAINS, NJ, 07950, US

(21) Appl. No.: 11/314,290

(22) Filed: Dec. 21, 2005

(51) Int. Cl. A61K 8/97; A61K 36/61

(52) U.S. Cl. 424/58; 424/742

(57) ABSTRACT

The present invention relates to oral care compositions containing select hydrocolloids and essential oils, wherein the undesirable taste of the essential oils is masked by the hydrocolloid.

The present invention relates to oral care compositions containing select hydrocolloids and essential oils, wherein the undesirable taste of the essential oils is masked by the hydrocolloid.

### BACKGROUND OF THE INVENTION

The present invention relates to oral care products comprising hydrocolloidal-masked essential oils. Oral compositions including mouthwashes and dentifrices containing essential oil compounds have been formulated using one or more of the following: menthol, methyl salicylate, eucalyptol and thymol are well known (U.S. Pat. No. 4,945,087; PCT Int. Appl. Nos. WO9416674; WO9407477; WO9418939, each of which is incorporated by reference in their entirety). These compositions often have an undesirable medicinal taste which can be unattractive to consumers. In particular, thymol tends to contribute the most to this undesirable taste. The combination of essential oils seems to intensify this undesirable taste.

It is desirable to have an oral care composition containing an essential oil mixture of menthol, eucalyptol, methyl salicylate, and thymol, wherein the undesirable taste of essential oil(s) are masked. This invention provides such compositions whose undesirable taste is effectively masked by using a hydrocolloid.

### SUMMARY OF THE INVENTION

The present invention relates to an oral care composition comprising:
a. an effective amount of hydrocolloid, wherein said hydrocolloid is selected from the group consisting of a plant extract, seaweed extract and mixtures thereof; and
b. about 0.01 weight % to about 5 weight % of essential oils, said essential oils comprising menthol, eucalyptol, methyl salicylate, and thymol, wherein the undesirable taste of the essential oils is masked by the hydrocolloid.

Another aspect of the present invention relates to an oral care composition comprising:
a. an effective amount of a hydrocolloid selected from the group consisting of an alginate or alginic acid derivative, pectin or pectin derivative, and mixtures thereof; and
b. about 0.01 weight % to about 5 weight % of essential oils, said essential oils selected from the group consisting of menthol, eucalyptol, methyl salicylate, and thymol, and mixtures thereof, wherein the undesirable taste of the essential oils is masked by the hydrocolloid.

Another aspect of the present invention relates to an oral care composition comprising:
a. about 0.01 weight % to about 1 weight % of propylene glycol alginate; and
b. about 0.01 weight % to about 5 weight % of essential oils, said essential oils selected from the group consisting of menthol, eucalyptol, methyl salicylate, and thymol, and mixtures thereof.

The present invention also relates to methods of using such compositions.

### DETAILED DESCRIPTION OF THE INVENTION

As used herein the term “comprising” means that the composition can contain other ingredients which are compatible with the composition and which preferably do not substantially disrupt the compositions of the present invention. The term encompasses the terms “consisting of” and “consisting essentially of”.

Unless otherwise indicated, all percentages and ratios used herein are by weight of the total composition. The term “wet weight” as referred to herein refers to the weight of undried material (i.e., liquid portion not evaporated off). All weight percentages, unless otherwise indicated, are on an active weight basis. All measurements made are at 25° C., unless otherwise designated.

The term “effective amount” as referred to herein refers to an amount of the oral care agent that is sufficient to at least reduce or relieve the condition, symptom, or disease being treated, but low enough to avoid any adverse side effects.

The term “taste masking” is difficult to quantify, but as referred to herein, it refers to a reduction of undesirable or unpleasant taste that would otherwise be present.

The compositions in this invention comprise oral rinses (e.g. mouth rinses or washes), dentifrices, oral gel liquids, gels, chewing gums, liquid center filled gums, mints, lozenges, edible films and the like having an effective concentration of essential oil compounds where the undesirable taste of the essential oils are masked by the addition of a hydrocolloid. Moreover, a less intense flavor is achieved in the compositions of this invention. In an aspect of the present invention, there is provided an oral care composition, a mouth wash, containing an effective amount of a hydrocolloid. Either the natural or synthetic form of these ingredients could be used in the composition of the present invention. Certain of these ingredients may provide a better masking effect of the essential oils in these compositions either alone or in combination with other hydrocolloid components.

Hydrocolloid

The compositions of the present invention comprise a hydrocolloid. Hydrocolloids typically encompass any substance which forms gels in water. Hydrocolloids useful in the present invention include natural, semi-synthetic, and synthetic types. The hydrocolloid can be for example, natural seaweed extract, natural seed gum, natural plant exudates, natural plant extracts, natural fiber extracts, biosynthetic gums, gelatins, biosynthetic process starch or cellulosic materials, alginates, carrageenans, guar, locust, tara, Arabic gum, ghatti gum, agar gum, xanthan gum, pectin, other like hydrocolloid source material or mixtures thereof. The hydrocolloid can be present at from about 0.01% to about 5% by weight. Generally, when the composition is a liquid, the hydrocolloid is present at from about 0.01% to about 0.3% by weight, optionally from about 0.01% to about 0.2% by weight of the total composition. Generally, when the composition is a dentifrice, the hydrocolloid is present at from about 0.05% to about 5% by weight, optionally from about 0.1% to about 3% by weight of the total composition. Generally, when the composition is a film, the hydrocolloid is present at from about 0.03% to about 3% by wet weight, optionally from about 0.05% to about 2% by wet weight.

In certain embodiments, the hydrocolloid is an alginate or alginic acid derivatives. Suitable alginates or alginic acid derivatives useful for the present invention, include, but are not limited to, calcium alginate, polypropylene alginate, potassium alginate, propylene glycol alginate, and sodium alginate and mixtures thereof. Optionally, the alginate is propylene glycol alginate (FMC BioPolymer). Alginate or alginic acid derivatives can be present at from about 0.01% to about 1% by weight, optionally from about 0.02% to about 0.05% by weight.

In certain embodiments, the hydrocolloid is pectin or pectin derivatives (GENU®). Suitable pectins useful for the present invention, include, but are not limited to pectin, low methoxyl pectin, calcium pectinate, sodium pectinate, potassium pectinate, and mixtures thereof. Pectin or pectin derivatives can be present at from about 0.05% to about 5% by weight, optionally from about 0.1% to about 1% by weight.

In certain other embodiments, the hydrocolloid is selected from a group consisting of polyvinylpyrrolidone (PVP®), carboxyvinylpolymers (Carbopol®), polyethylene oxide polymers (Polyox®), and mixtures thereof In further embodiments, the hydrocolloid is selected from a group consisting of carboxymethylcellulose, methylcellulose, hydroxypropylcellulose, hydroxypropylmethylcellulose, and mixtures thereof.

Essential Oil

Also useful in the composition of the present invention are essential oils. In certain embodiments of the present invention, the essential oil provides biologic or therapeutic activity, especially antimicrobial activity, in the oral cavity. In one embodiment the essential oils are selected from a group consisting of menthol, methyl salicylate, eucalyptol, thymol and mixtures thereof. Thymol is generally considered to have good antimicrobial activity. Thymol is also an anthelmintic and an antiseptic. Generally, when the composition is a liquid, then the total amount of essential oils present in the liquid composition of the present invention can be from about 0.05 to about 0.35% by weight, optionally with about 0.12 to about 0.28% by weight of the total composition. The total amount of essential oils present in the liquid compositions of the present invention can vary as long as they are in amounts sufficient to provide antimicrobial efficacy. The eucalyptol is generally present in amounts of about 0.07 to about 0.11% by weight and optionally about 0.08 to about 0.10% by weight; menthol is generally present in amounts of about 0.03 to about 0.06% by weight and optionally about 0.04 to about 0.05% by weight; methyl salicylate is generally present in amounts of about 0.03 to about 0.08% by weight and optionally about 0.04 to about 0.07% by weight, thymol is generally present in amounts of about 0.03 to about 0.08% by weight and optionally about 0.04 to about 0.07% by weight based on the total volume of the composition. Generally, when the composition is a film, the total amount of essential oils present in the film composition of the present invention can be from about 0.05 to about 15% by wet weight, optionally with about 0.20 to about 10% by wet weight of the total composition. The total amount of essential oils present in the film compositions of the present invention can vary as long as they are in amounts sufficient to provide antimicrobial efficacy. Generally the amount of thymol, methyl salicylate and eucalyptol is from about 0.01 to about 4% by wet weight of the film composition, optionally about 0.50 to about 3.0% by wet weight, and optionally about 0.70 to about 2.0% by wet weight of the film. Menthol is generally added from about 0.01 to about 15% by wet weight of the composition, optionally about 2.0 to about 10% by wet weight, and optionally from about 3 to about 9% by wet weight of the film. Generally, when the composition is a dentifrice, the total amount of essential oils present in the dentifrice composition of the present invention can be from about 0.1 to about 4% by weight, optionally with about 0.5 to about 3% by weight of the total composition. The total amount of essential oils present in the dentifrice compositions of the present invention can vary as long as they are in amounts sufficient to provide antimicrobial efficacy. Menthol may be in the dentifrice composition of the present invention in an amount of from about 0.01 to about 1.0% by weight, optionally in an amount of from about 0.10 to about 0.7% by weight of the composition. Eucalyptol may be in the dentifrice composition of the present invention in an amount of from about 0.01% to about 1.0% by weight, optionally in an amount of from about 0.05% to about 0.5% by weight. Methyl salicylate may be in the dentifrice composition of the present invention in an amount of from about 0.01% to about 1.0% by weight, optionally in an amount of from about 0.04% to about 0.6% by weight. Thymol may be in the dentifrice composition of the present invention in an amount of from about 0.01% to about 1.0% by weight, optionally in an amount of from about 0.1% to about 0.6% by weight.

In certain embodiments of the present invention, the ratio of said essential oil to hydrocolloid is from about 20:1 to about 1:5, optionally from about 10:1 to about 1:3, optionally from about 5:1 to about 1:1 by weight of the total composition. Certain embodiments of the present invention incorporates hydrocolloids in the form of an aqueous solution having a viscosity within the range of about 1-40 centipoise, optionally about 2-10 centipoise, or optionally about 3-5 centipoise (Brookfield RVT, #5 RV Spindle, 10 rpm, 25° C.).

Without being limited by theory, it is believed that the hydrocolloid provides a coating in the oral cavity that assists in taste-masking essential oil. This coating is believed to reduce the perceived undesirable taste of the essential oil in the oral cavity. Additionally or alternatively, it is believed that the hydrocolloid can associate with the essential oil such that the perceived undesirable taste of the essential oil is reduced, yet the bio-availability of the essential oil is retained in the oral cavity.

Other useful essential oils, alone or in combination with the above essential oils, include carvacrol, camphor, anethole, carvone, eugenol, isoeugenoi, limonene, osimen, n-decyl alcohol, citronel, a-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, cassia oil, clove oil, coal tar, eucalyptus oil, guaiacol, tropolone derivatives such as hinokitiol, lavender oil, mustard oil, phenol, phenyl salicylate, pine oil, pine needle oil, sassafras oil, spike lavender oil, storax, thyme oil, tolu balsam, terpentine oil, clove oil, and mixtures thereof.

Optional Ingredients

Humectants in oral products of the present invention impart to the mouth a moist and elegant feel and, if incorporated at sufficient concentration, may further inhibit the harshness of the essential oils in these compositions. Some humectants, for example, can provide sweetness to the composition, as well. Suitable humectants include edible polyhydric alcohols such as glycerin, sorbitol, propylene glycol, butylene glycol, xylitol and cyclodextrins, including their derivatives. A humectant generally is present in an amount ranging from about 0.1 weight % to about 30 weight % for oral rinses and from about 10 weight % to about 50 weight % of the total composition for dentifrice and oral gel compositions.

Oral surfactants useful in the present invention include certain nonionic, anionic and amphoteric surfactants. The oral surfactants include block co-polymers of polyoxyethylene and polyoxypropylene such as the Pluronics from BASF. Other oral surfactants include soluble alkyl sulfonates having 10 to 18 carbon atoms and sulfates of monoglycerides of fatty acids having 10 to 18 carbon atoms or sarcosinates (including salts and derivatives) such as sodium-N-lauroyl sarcosinate. Amphoteric surfactants that can be used include betaines, sulfobetaines and amidobetaines such as the TEGO betaines from Goldschmidt Chemical Corporation. Mixtures of anionic, nonionic and amphoteric surfactants can be used. These ingredients are generally present from about 0.01 weight % to about 10 weight %, optionally from about 0.01 weight % to 1 weight % for oral rinses and from about 0.5 weight % to about 2 weight % of the total composition for dentifrices and oral gels.

For dentifrice and oral gel compositions, abrasives may also be added. Suitable abrasives include precipitated silica or silica gels which have an average particle size ranging from about 0.1 to about 50 microns. Silica abrasives include those marketed under the trade name “Sylodent” or “Syloid” by the W. R. Grace & Co. and those marketed under the trade name “Zeodent” by the J. M. Huber Corp. Other suitable abrasives, having a suitable particle size as described above, include .beta.-phase calcium pyrophosphate, alumina and calcium carbonate. The amount of abrasive in a dentifrice composition ranges up to about 60 weight %, optionally from about 10 weight % to about 40 weight % of the total composition.

Oral rinse, dentifrice, and oral gel compositions of the present invention may also contain a suitable fluoride source. Typical sources include soluble salts of the fluoride ion (e.g. sodium fluoride, potassium fluoride, stannous fluoride, stannous fluorozirconate) or, soluble salts of the monofluorophosphate ion (e.g. sodium monofluorophosphate). Optionally, the fluoride source is sodium fluoride. The fluoride ion source should provide from about 50 ppm to about 2,500 ppm fluoride, optionally from about 250 ppm to about 1500 ppm for dentifrice and oral gel compositions, and from about 50 ppm to about 250 ppm fluoride for oral rinses.

Antiplaque agents can be optionally added to the compositions of the present invention. These include cetyl pyridinium chloride and related quatemary salts such as chlorhexidine, zinc salts such as zinc chloride, stannous salts such as stannous chloride, or stannous fluoride and peroxygens such as hydrogen peroxide, carbamide peroxide, sodium percarbonate, magnesium perphthalate or sodium perborate. These optional antiplaque agents are generally present at levels less than about 5 weight %.

Anticalculus agents can be optionally added to the compositions of the present invention. These include tetra-alkali metal pyrophosphate salts and zinc salts, such as zinc chloride. These optional anticalculus agents are generally present at levels less than about 5 weight % for pyrophosphate salts and less than about 3 weight % for zinc salts.

Additionally, an embodiment of this invention will contain flavors. These flavors will contain ingredients including, for example, vanilla, spearmint, mint, citrus, and/or cinnamon. The flavors will be used in amounts from about 0.05 to about 1.0% by weight based on the total composition.

In compositions of the present invention, preservatives may be used, especially in non-alcohol or low alcohol compositions. These include benzoic acid, sodium benzoate, methylparaben, propylparaben, sorbic acid and potassium sorbate. These preservative agents are generally present at levels less than about 2 weight %.

In compositions relating to the invention, buffering systems may be used to stabilize the pH in the product. The pH of the oral rinse, dentifrice, and oral gel compositions can range from about 3.5 to about 8.5. Typical buffering systems include, but are not limited to, citrate, benzoate, gluconate and phosphate. Buffering systems are present in concentrations from about 0.01 weight % to about 1 weight %.

Thickening agents or binders are an optional component of the compositions. Typical thickening include, xanthan gum, carragenan, carboxyvinyl polymers, carbomers, cellulose gums such as carboxymethyl cellulose, cellulose derivatives such as hydroxyethylcellulose and silicas. Thickeners are usually present in the compositions less than about 2 weight % in oral rinses. In dentifrices and oral gels, silica-based thickeners can be used at concentrations less than about 20 weight %. “Sylox” or “Sylodent” by W. R. Grace & Co. is the trade name of the silica-based thickener, xanthan gum.

The sugar alcohol which may be used in this invention may include, for example, sorbital, xylitol, lactitol, mannitol, maltilol, hydrogenated starch hydrolsate, erythritol, reducing paratinose and mixtures thereof. Sugar alcohols are generally present in an amount ranging from about 0.001 to about 5 weight % for oral rinse, dentifrice and oral gel compositions. Additionally, orally acceptable sweetening agents such as saccharin, sucralose, lactose, maltose, aspartame, sodium cyclamate, and polydextrose and mixtures thereof can also be added to the present compositions. Sweetening agents also are generally present in an amount ranging from about 0.001 to about 5 weight % for oral rinse, dentifrice and. oral gel compositions. Orally acceptable coloring agents generally are present in an amount less than about 0.01 weight %.

### EXAMPLES

The following examples further describe and demonstrate embodiments within the scope of the present invention. The examples are given solely for the purpose of illustration, and are not to be construed as limitations of the present invention since many variations thereof are possible without departing from its scope.

### Example 1

A mouthwash having the following components was prepared:

| INGREDIENT | % Wet | |
|---|---|---|
| Propylene Glycol Alginate | 0.03000 | w/v |
| Alcohol USP, Ethanol | 22.66900 | w/v |
| Poloxamer 407 | 0.25000 | w/v |
| Benzoic acid, USP | 0.12000 | w/v |
| Thymol NF | 0.06390 | w/v |
| Eucalyptol USP | 0.09220 | w/v |
| Menthol USP | 0.04002 | w/v |
| Methyl Salicylate NF | 0.06600 | w/v |
| Flavor | 0.13689 | w/v |
| Sodium Benzoate NF | 0.03540 | w/v |
| Sucralose | 0.05000 | w/v |
| Sorbital Solution USP | 20.00000 | w/v |
| FD&C Blue No. 1 | 0.00006 | w/v |
| FD&C Yellow No. 6/E110 | 0.00009 | w/v |
| Purified Water, USP0EP | q.s. | |
| Total | 100.00000 | w/v |

Propylene Glycol Alginate was sprinkled into the vortex of approximately 300 ml of USP water. It was then mixed for approximately 10 minutes in a covered beaker until the alginate dissolved. In the primary container, Alcohol USP, Menthol, Thymol, Poloxamer 407, Benzoic acid, Methyl Salicylate, Eucalyptol, and flavor were added to alcohol and mixed well. Approximately 100 ml of purified water was added to the primary container. Sorbitol solution was added to the primary container and mixed well. Protanal ester solution from the covered beaker was added and mixed well. Sodium Benzoate, Sucralose, Yellow #6 and Blue #1 were added and mixed until clear. The volume Was adjusted to I liter with balance of purified water and mixed well. The total contents were then filtered through a 45 micron filter and stored.

### Example 2

A mouthwash having the following components was prepared:

| INGREDIENT | % Wet | |
|---|---|---|
| Pectin | 0.1500 | w/v |
| Alcohol USP, Ethanol | 22.7215 | w/v |
| Poloxamer 407 | 0.2500 | w/v |
| Benzoic acid, USP | 0.1161 | w/v |
| Thymol NF | 0.0639 | w/v |
| Eucalyptol USP | 0.0922 | w/v |
| Menthol USP | 0.0407 | w/v |
| Methyl Salicylate NF | 0.0660 | w/v |
| Flavor | 0.3390 | w/v |
| Sodium Benzoate NF | 0.0400 | w/v |
| Sucralose | 0.0500 | w/v |
| Sorbital Solution USP | 20.0000 | w/v |
| FD&C Blue No. 1 | 0.0000 | w/v |
| FD&C Yellow No. 6/E110 | 0.0001 | w/v |
| Purified Water, USP/EP | q.s. | |
| Total | 100.0000 | w/v |

200 ml of USP water was placed into a 400 ml beaker. Pectin USP was then sprinkled into the 200 ml of USP water. The beaker was covered with aluminum foil. The solution was mixed for 20 minutes with a Lightning Mixer at 500 RPM. In the primary container, alcohol USP, menthol, thymol, poloxamer 407, and benzoic acid were mixed until clear. Methyl salicylate, eucalyptol and flavors were added to the primary container. Approximately 150 ml of purified water was added to the primary container. Sorbitol solution was added to the primary container and mixed well. The pectin solution from the covered beaker was added and mixed well. Purified water was added to the solution to attain 80% of the final volume and mixed well. Sodium Benzoate, Sucralose, Yellow #6 and Blue #1 were added and mixed until clear. The total contents were then filtered through a 45 micron filter and stored.

### Example 3

An edible film having the following components was prepared:

| INGREDIENT | % Wet | |
|---|---|---|
| Pullulan | 15.534 | w/w |
| Propylene Glycol Alginate | 1.000 | w/w |
| Copper Gluconate | 0.361 | w/w |
| Acesulfame Potassium Salt | 0.508 | w/w |
| Atmos 300 | 0.355 | w/w |
| Flavor | 2.500 | w/w |
| Thymol NF | 0.300 | w/w |
| Eucalyptol USP | 0.186 | w/w |
| Menthol USP | 2.010 | w/w |
| Methyl Salicylate NF | 0.186 | w/w |
| Polysorbate 80 | 0.355 | w/w |
| Carrageenan | 0.348 | w/w |
| Sucralose | 1.011 | w/w |
| Locust Bean Gum | 0.069 | w/w |
| Xanthan Gum | 0.035 | w/w |
| FD&C Yellow No. 6 | 0.005 | w/w |
| Purified Water, USP/EP | 75.239 | w/w |
| Total | 100.000 | w/w |

Water was weighed and placed in a container. Yellow #6, Copper Gluconate, Acesulfame Potassium Salt, and Sucralose was added to the water and mixed for 30 minutes. In a separate container, Xanthan Gum, Locust Bean Gum, Carrageenan, Propylene Glycol Alginate, and Pullulan were mixed (dry) until uniform. This dry-mix was slowly added to the water and mixed for about 1 to 2 hours (until the gums were hydrated). In a separate container, Flavor, Thymol, Methyl Salicylate, Eucalyptol, Menthol, Polysorbate 80, and Atmos 300 was weighed and mixed until dissolved and uniform. This mixture was then added to the water and mixed well until uniform. The mixture was then poured and cast to a desired film thickness at room temperature. The film was then dried under warm air, cut to dimension, and packaged.

### Example 4

A dentifrice having the following components was prepared:

| INGREDIENT | % Wet | |
|---|---|---|
| Carboxymethyl Cellulose | 1.00 | w/w |
| Pectin | 3.00 | w/w |
| Silica | 22.00 | w/w |
| Sodium Lauryl Sulfate | 1.50 | w/w |
| Sodium Phosphate dibasic | 0.80 | w/w |
| Flavor | 1.00 | w/w |
| Thymol NF | 0.64 | w/w |
| Eucalyptol USP | 0.92 | w/w |
| Menthol USP | 0.44 | w/w |
| Sodium Saccharin | 0.50 | w/w |
| Titanium dioxide | 1.00 | w/w |
| Glycerin | 53.00 | w/w |
| Purified Water, USP/EP | 14.00 | w/w |
| Total | 100.000 | w/w |

The jacket temperature of a mixing tank was set to heat up to about 70° C. As the tank heated, the glycerin and water were added to the mixing tank and agitation was started. Once the temperature reached approximately 50° C., in the heating process, the sodium fluoride, sodium saccharin, tripolyphosphate, and titanium dioxide were added. In a separate container, the carboxy methyl cellulose, pectin, xanthan gum, and silica were added and mixed. The pectin mixture was then added to the mixing tank with high agitation. The sodium lauryl sulfate was added to the combination and mixing was continued. The tank was cooled to 50° C., and then the essential oils and the flavor was added. Mixing continued for approximately 30 minutes. The combination was allowed to cool to room temperature, and the toothpaste was packaged into toothpaste tubes.

What is claimed is:

1. An oral care composition comprising:
a. an effective amount of a hydrocolloid, wherein said hydrocolloid is selected from the group consisting of a plant extract, seaweed extract and mixtures thereof; and
b. about 0.01 weight % to about 5 weight % of essential oils, said essential oils comprising menthol, eucalyptol, methyl salicylate, and thymol, wherein the undesirable taste of the essential oils is masked by the hydrocolloid.

2. The oral care composition of claim 1, wherein the seaweed extract is an alginate or alginic acid derivative.

3. The oral care composition of claim 2, wherein the alginate or alginic acid derivative is selected from the group consisting of calcium alginate, polypropylene alginate, potassium alginate, propylene glycol alginate, and sodium alginate.

4. The oral composition of claim 1, wherein the plant extract is a pectin or pectin derivative.

5. The oral composition of claim 4, wherein the pectin or pectin derivative is selected from the group consisting of pectin, low methoxyl pectin, calcium pectinate, sodium pectinate, potassium pectinate, and mixtures thereof.

6. The oral composition of claim 1, wherein the essential oils to hydrocolloid ratio is at least about 20:1 to about 1:5 by weight.

7. The oral composition of claim 1 further comprising about 0.01 weight % to about 10 weight % of an orally acceptable surfactant.

8. The oral composition of claim 1 further comprising about 0.01 weight % to about 1 weight % of a flavoring agent.

9. The oral composition of claim 1 further comprising at least one sugar alcohol.

10. The oral composition of claim 9, wherein said sugar alcohol is selected from the group consisting of sorbital, xylitol, lactitol, mannitol, maltilol, hydrogenated starch hydrolsate, erythritol, reducing paratinose and mixtures thereof.

11. The oral composition of claim 1 further comprising a humectant.

12. The oral composition of claim 1 in a form selected from the group consisting of toothpaste, mouthwash, gel, toothpowder, edible film, film forming dentifrice, chewing gum, tablet, capsule, mouth spray and lozenge.

13. An oral care composition comprising:
a. an effective amount of a hydrocolloid selected from the group consisting of an alginate or alginic acid derivative, pectin or pectin derivative, and mixtures thereof; and
b. about 0.01 weight % to about 5 weight % of essential oils, said essential oils selected from the group consisting of menthol, eucalyptol, methyl salicylate, and thymol, and mixtures thereof, wherein the undesirable taste of the essential oils is masked by the hydrocolloid.

14. An oral care composition comprising:
a. about 0.01 weight % to about 1 weight % of propylene glycol alginate; and
b. about 0.01 weight % to about 5 weight % of essential oils, said essential oils selected from the group consisting of menthol, eucalyptol, methyl salicylate, and thymol, and mixtures thereof.

15. An oral care composition 14 in a form selected from the group consisting of toothpaste, mouthwash, gel, toothpowder, edible film, film forming dentifrice, chewing gum, tablet, capsule, mouth spray and lozenge.

16. A method for masking the undesirable taste of essential oils in an oral composition comprising adding an effective amount of a hydrocolloid, wherein said hydrocolloid is a plant extract or seaweed extract, and wherein the essential oils to hydrocolloid ratio is at least about 20:1 to about 1:5 by weight.

17. The method of claim 16, wherein the seaweed extract is an alginate or alginic acid derivative.

18. The method of claim 17, wherein the seaweed extract is an alginate or alginic acid derivative selected from the group consisting of calcium alginate, polypropylene alginate, potassium alginate, propylene glycol alginate, and sodium alginate.

19. The method of claim 16, wherein the plant extract is a pectin or pectin derivative.

20. The method of claim 19, wherein the plant extract is a pectin or pectin derivative selected from the group consisting of pectin, low methoxyl pectin, calcium pectinate, sodium pectinate, potassium pectinate, and mixtures thereof.
